(19) United States
(12) Patent Application Publication
(10) Pub. No.: US 2021/0113439 A1
(43) Pub. Date: Apr. 22, 2021

(54) Fluoride-free Oral Care Composition

(71) Applicant: John McNeil, New York City, NY (US)

(72) Inventor: John McNeil, New York City, NY (US)

(21) Appl. No.: 17/067,650

(22) Filed: Oct. 10, 2020

Related U.S. Application Data
(60) Provisional application No. 62/973,675, filed on Oct. 18, 2019.

(51) Int. Cl. A61K 8/19; A61K 8/9794; A61K 8/60; A61K 8/9789; A61K 8/67; A61Q 11/00

(52) U.S. Cl. CPC A61K 8/19; A61K 8/9794; A61K 8/602; A61Q 11/00; A61K 8/673; A61K 8/67; A61K 8/678; A61K 8/9789

(57) ABSTRACT

The present invention relates to toothpaste compositions for oral care, and more particularly relates to natural Fluoride-free toothpaste composition. The natural fluoride-free oral care composition comprises of charcoal, coconut oil, baking soda, natural sweetener such as stevia powder, peppermint, cinnamon, and myrrh. In the present composition, peppermint, cinnamon, and myrrh exhibit synergistic antioxidant activity with better potential effects than other commercial toothpastes available in the market. The activated charcoal provides higher adsorptive strength and makes it capable of removing all unnecessary tooth surface adhesive substances.

### CROSS-REFERENCE TO RELATED APPLICATIONS

For purposes of the USPTO extra-statutory requirements, the present application constitutes a nonprovisional patent application of U.S. provisional patent application Ser. No. 62/973,675, naming John McNeil as inventor, filed Oct. 18, 2019.

### TECHNICAL FIELD OF THE INVENTION

The present invention relates to toothpaste compositions for oral care, and more particularly relates to natural Fluoride-free toothpaste composition.

### BACKGROUND OF THE INVENTION

Now a day's oral hygiene is essential for preserving teeth and preventing various diseases related to bacteria and plaque buildup in the mouth. Tooth hypersensitivity is a common problem which affects about tens of millions of people. Moreover, as parents increasingly worry about the types of products being ingested by children and due to consumption of such products leads to the development of infectious bacteria that further causes unhealthy oral condition.

A large variety of oral care products exist on the market, that whitens and cleans the surface of the teeth, freshens breath, and sanitizes the mouth, which is predominantly include synthetic detergents as surfactants, and additional chemicals, such as natural or synthetic fluoride.

Although these ingredients, may efficiently remove grime and protect enamel, but they have been known in some instances to cause ulcers in the mouth and may pose other risks when ingested. Moreover, in the case of children oral hygiene, parents increasingly worry about the types of products being ingested by children, and more and more parents are attempting to use natural toothpaste for their children.

In view of this, various solutions have been proposed, none combine the characteristics of the present invention. In recent years, as consumers become more concerned about their health and the types of chemicals entering into their bodies, the natural ingredients-based toothpastes have experienced a small revival amongst a select group of consumers. However, to date, most consumers are dissatisfied with the overall performance of natural toothpastes. In addition, in the case of children, it is difficult to get children to regularly use natural toothpaste due to the roughness/texture, blunt taste, and grittiness of the toothpaste.

Thus, there is a need for a natural, fluoride-free oral care composition that contains charcoal, coconut oil, baking soda, stevia powder, peppermint, cinnamon, and myrrh. It is packed with vitamins that help to protect teeth.

### SUMMARY OF THE INVENTION

Embodiments of the invention solve the above-mentioned problems by providing a unique oral care composition. The natural fluoride-free oral care composition comprises charcoal, coconut oil, baking soda, natural sweetener such as stevia powder, peppermint, cinnamon, and myrrh. In the present composition, peppermint, cinnamon, and myrrh exhibit synergistic antioxidant activity with better potential effects than other commercial toothpastes available in the market. Additionally, this composition comprises of vitamins that help to protect teeth. Furthermore, the activated charcoal provides higher adsorptive strength and makes it capable of removing all unnecessary tooth surface adhesive substances. Specifically, this property makes its use more effective than traditional charcoal as a tooth-cleaning agent.

This summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other aspects, and advantages of the subject matter described herein will become apparent from the following detailed description, and claims.

### BRIEF DESCRIPTION OF THE DRAWINGS

Drawings not required as the subject matter of the present invention is related to a composition.

### DETAILED DESCRIPTION OF THE INVENTION

The following detailed description are intended to describe aspects of the invention in sufficient detail to enable those skilled in the art to practice the invention. Other embodiments can be utilized, and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, embodiments of the invention can include a variety of combinations and/or integrations of the embodiments described herein.

The present invention comprises a charcoal, an antibacterial agent coconut oil charcoal, coconut oil, baking soda, natural sweetener such as stevia powder, peppermint, and myrrh. Additionally, the composition also includes vitamins that provides additional protection to the teeth.

In one embodiment, the present invention comprises an activated charcoal, which aid to remove and absorb surface stains on teeth, provides higher adsorptive strength and makes it capable of removing all unnecessary tooth surface adhesive substances. Specifically, this property makes its use more effective than traditional charcoal as a tooth-cleaning agent.

In the same embodiment, the composition further comprises coconut oil as an antibacterial. The coconut oil attracts bacteria and when the toothpaste is spitted out, the coconut oil that has attracted bacteria, expels out along with bacteria. Moreover coconut oil also acts as natural moisturizer and helps to keep mouth moist.

In the same embodiment, the composition further comprises baking soda, one of the components for cleaning agent. Baking soda acts as an abrasive, which help in removing plaque from the surface of the teeth.

In the same embodiment, the composition further comprises stevia powder as a natural sweetener and flavor enhancer. Stevia powder is used as a healthy alternative to saccharin in toothpaste. It is also useful as an anti-inflammatory, flavor enhancer and antioxidant.

In the same embodiment, the composition further comprises peppermint as a flavoring agent.

In the same embodiment, the composition further comprises cinnamon act as powerful antioxidant. Also useful as a flavor enhancer and anti-inflammatory.

In the same embodiment, the composition further comprises myrrh, which is also act as an antioxidant.

In the same embodiment, the composition further comprises vitamin B5, vitamin B6, vitamin B5, vitamin D, vitamin E that help to protect the teeth.

In the same embodiment, peppermint cinnamon, and myrrh exhibit synergistic antioxidant activity with better potential than other commercial toothpaste available in the market.

In a preferred embodiment, the natural, fluoride-free oral care composition comprising: a cleansing agent charcoal in the range of 20-60% (w/w); an antibacterial agent coconut oil in the range of 5-15% (w/w); an abrasive baking soda in the range of 10-25% (w/w); a natural sweetener stevia powder in the range of 0.03-3% (w/w); a flavor enhancer peppermint in the range of 2-5% (w/w); an anti-inflammatory agent cinnamon in the range of 1-5% (w/w); an antioxidant myrrh in the range of 1-5% (w/w); vitamin B5 in the range of 0.1-1% (w/w); vitamin B6 in the range of 0.1-1% (w/w); vitamin Din the range of 0.1-1% (w/w); vitamin E in the range of 0.1-1% (w/w); wherein the composition has a stable viscosity; and wherein the composition is in the form of a toothpaste.

The present invention has numerous advantages in comparison with the other marketed products such as anti-inflammatory, antibacterial, full of antioxidants, free from harmful synthetic ingredients, with pleasant taste. Furthermore, the present composition provides overall improved oral health. Specifically, ingredients which are composed in the present composition are responsible to brighten the teeth in a natural way.

Although the description above contains many specifics, these should not be construed as limiting the scope of the embodiments but as merely providing illustrations of some of several embodiments.

Having thus described in various embodiments of the invention, what is claimed as new and desired to be protected by Letters Patent includes the following:

1. A natural, fluoride-free oral care composition comprising:
a. charcoal in the range of 20-60% (w/w);
b. coconut oil in the range of 5-15% (w/w);
c. baking soda in the range of 10-20% (w/w);
d. stevia powder in the range of 0.03-3% (w/w);
e. peppermint in the range of 2-5% (w/w);
f. cinnamon in the range of 1-5% (w/w);
g. myrrh in the range of 1-5% (w/w);
h. vitamin B5 in the range of 0.1-1% (w/w);
i. vitamin B6 in the range of 0.1-1% (w/w);
j. vitamin D in the range of 0.1-1% (w/w); and
k. vitamin E in the range of 0.1-1% (w/w);

2. The natural, fluoride-free oral care composition of claim 1, wherein the composition has a stable viscosity; and wherein the composition is in the form of a toothpaste.

3. The natural, fluoride-free oral care composition of claim 1, where in the peppermint, cinnamon, and myrrh exhibit synergistic antioxidant activity.

4. The natural, fluoride-free oral care composition of claim 1, where in the charcoal aids to remove and absorb surface stains on teeth, provides higher adsorptive strength and removes all unnecessary tooth surface adhesive substances.

5. The natural, fluoride-free oral care composition of claim 1, where in the coconut oil attracts bacteria and when the toothpaste is spitted out, the coconut oil that has attracted bacteria, expels out along with bacteria.

6. The natural, fluoride-free oral care composition of claims 1 and 4, where in the coconut oil also acts as natural moisturizer and helps to keep mouth moist.

7. The natural, fluoride-free oral care composition of claim 1, where in the baking soda removes plaque from the surface of the teeth.

8. The natural, fluoride-free oral care composition of claim 1, where in the stevia powder acts as an anti-inflammatory, flavor enhancer and antioxidant.
